(19) United States
(12) Patent Application Publication
(10) Pub. No.: US 2013/0231635 A1
(43) Pub. Date: Sep. 5, 2013

(54) MULTIVOLUME CARTRIDGE ASSEMBLY AND METHOD OF MAKING THE SAME

(71) Applicant: Gannon Lee, Encinitas, CA (US)

(72) Inventor: Gannon Lee, Encinitas, CA (US)

(21) Appl. No.: 13/779,570

(22) Filed: Feb. 27, 2013

Related U.S. Application Data
(60) Provisional application No. 61/605,586, filed on Mar. 1, 2012.

(51) Int. Cl. A61M 5/19; A61J 1/20

(52) U.S. Cl. CPC A61M 5/19; A61J 1/20; A61J 1/2093
USPC 604/506; 604/403; 604/416

(57) ABSTRACT

A multivolume cartridge assembly having two or more internal compartments for storing and administering one or more medicinal compounds. In some embodiments, a second internal volume is positioned in the distal end of the cartridge assembly, and in other embodiments, the second internal volume is positioned in the apical end of the cartridge assembly. In some embodiments, the cartridge is configured to allow for long term storage of two or more medicinal compounds without mixing of the two or more compounds during storage. The cartridge, in some embodiments, utilizes a stopper assembly to create fluid communication between the two or more internal compartments of the cartridge and allow for mixing of the contents of the two or more compartments prior to the administration of those contents to a patient.

### RELATED APPLICATIONS

Any and all priority claims identified in the Application Data Sheet, or any correction thereto, are hereby incorporated by reference under 37 CFR 1.57.

### TECHNICAL FIELD

The disclosure relates generally to the field of medicinal containers. More specifically, aspects of the disclosure relate to multivolume medicinal containment and administration systems and processes, wherein two or more compounds may be stored separately in a single container before use and may be mixed prior to administering the compounds to a patient.

### BACKGROUND

A container in which medicinal compounds are delivered is in certain instances herein referred to as a carpule. While the term “carpule” is sometimes associated with a specific container used in dental anesthesia procedures, the features and embodiments of the “carpule” described in the present application could be applied to other containers and/or in other fields of medicine and dentistry. A carpule has two ends. The end from which the medication is expunged and delivered to the patient will be referred to as the apical end. The opposite end of the carpule will be referred to as the distal end.

Carpules can be used in connection with dental anesthetics. Some carpules have the apical end covered with a thin silicone barrier and a metallic clamp which prevents the barrier from movement and dislodgment. The clamp does not come into contact with the anesthetic. Only the silicone barrier, as well as the material of the carpule walls, comes into contact with the anesthetic at the apical end. The apical end of the carpule has an indentation so as to allow the clamp to seal around the material of the carpule. On the distal end, a rubber stopper is placed in the carpule, which seals the anesthetic in the center between the apical barrier and the apical end of the stopper. This stopper is allowed to move axially from distal to apical and vice versa.

When administering anesthetic to a patient, a carpule can be placed into a dental syringe. The syringe has an expunging part on the distal end that, when engaged into the bottom of the stopper, allows the practitioner to push the anesthetic toward the apical end of the carpule. The distal end of the syringe that moves from the distal toward the apical end is called the harpoon. The harpoon has sharp edges that engage into the rubber stopper, allowing the practitioner to move the stopper apically and distally. A needle is inserted and attached to the apical end of the syringe assembly, which contacts and punctures the silicone barrier of the carpule. Current carpule design for anesthetics allows for only one homogenous solution in the carpule.

### SUMMARY OF THE DISCLOSURE

The disclosure provides for separate storage and mixing capabilities in a single carpule or container and includes embodiments of a multivolume cartridge assembly for administering medicinal compounds, methods of manufacturing the multivolume cartridge, and a method for using the multivolume cartridge. The multivolume cartridge design, as disclosed herein, is configured to allow for the administration of one or more medicinal compounds to a patient, and to allow for mixing of two or more compounds prior to administration if desired. In some embodiments, the multivolume cartridge can be cylindrical in shape. The multivolume cartridge can also be a rectangular prism, triangular prism, or any similar shapes. The multivolume cartridge can be sized and shaped to fit within or otherwise interface with a standard medicinal or dental syringe assembly.

There is a need for a cartridge design for anesthetics that allows for the storage and administering of two or more medicinal compounds in two or more compartments. For example, a common anesthetic used in the dental sciences is Lidocaine 2% with 1:100,000 epinephrine. The pH of this solution is very low (e.g. a pH of 3.4-4.5) due to the vasopressor (epinephrine) in the local anesthetic. The low pH, and thus high acidity of the solutions “stings” on injection. This low pH can also cause tissue injury and cause the anesthetic to have a relatively slow onset. Additionally, the local anesthetic could not work reliably in the presence of infection and inflammation, as is the case in a root canal treatment. The epinephrine is useful, however, for increasing the efficacy and length of effectiveness of the anesthetic. One solution which can allow for use of the epinephrine while reducing some of its negative side effects is to buffer the local anesthetic with sodium bicarbonate, thereby increasing the pH of the anesthetic solution. In doing this, carbon dioxide is introduced into the compound. For example, mixing one part sodium bicarbonate 8.4% with 10 parts of the Lidocaine solution can result in a solution pH of 7.3-7.4. This lessens the “sting,” reduces tissue injury, and increases the onset speed of the anesthetic.

The multivolume cartridge assembly, in some embodiments, comprises a container having an apical and distal end. The apical end has an opening which can be sealed with a silicone barrier or other sealing means. The silicone barrier or other sealing means can be held in place with a clamp or other adhering means. The apical end can have an indentation in the walls of the container. The indentation can be shaped so as to allow for a needle assembly to be attached to the apical end of the cartridge. Additionally or alternatively, the apical end of the container can have threading which can be configured to engage with a needle assembly.

The distal end of the cartridge has an opening which can be sealed with a stopper or stopper assembly. Said stopper can be made of rubber or some other material suitable for sealing the distal end of the cartridge, such as butyl rubber or silicone rubber. Said stopper assembly may further comprise an interior enclosure attached to the apical end of the stopper. The walls of the interior enclosure may be made of silicone or some other flexible material suitable for preventing liquid or gas from entering the interior enclosure from outside the interior enclosure. The interior enclosure could house a solid or liquid medicinal compound. The stopper assembly may further comprise a tab assembly which may be used to create an opening in the wall of the interior enclosure.

In an embodiment of the multivolume cartridge assembly, the tab assembly comprises a small strip tab with at least one hole in it. The tab can be constructed of silicone, silicone-coated thick paper or some other flexible material which is resistant to deterioration in a liquid environment. The tab can be pushed or pulled from the distal end of the cartridge assembly so that the at least one hole in the tab can line up with at least one hole in the interior enclosure and create an opening in the wall of the interior enclosure. In another embodiment of the multivolume cartridge assembly, the tab assembly comprises a semi-rigid flexible tab with a sharp apical end. The distal end of the tab can be pushed (and in some embodiments, pulled) so that the sharp apical end can pierce the wall of the interior enclosure and create an opening in the wall of the interior enclosure. In another embodiment of the multivolume cartridge assembly, the tab assembly comprises a small strip tab that could have a small plug on it that could engage with a hole in the wall of the interior enclosure. This strip tab can be pulled so that the plug can disengage from the hole in the wall of the interior enclosure and create an opening in the wall of the interior enclosure.

In other embodiments of the multivolume cartridge assembly, an interior enclosure can be formed on the apical end of the cartridge. Tab assemblies could be utilized, as in the distal interior enclosure assemblies, to create an opening or openings in the wall of the interior enclosure.

In some embodiments of the multivolume cartridge assembly, an exterior enclosure could be formed between the outer walls of an interior enclosure and the inner walls of the containing vessel. The exterior enclosure could house a solid or liquid medicinal compound. In some embodiments, a tab assembly could be used to create an opening in the walls of interior enclosure and allow mixing between the compounds in the interior and exterior enclosures. Said mixing could be done before administering the medicinal compound or compounds to a patient.

A cartridge assembly with a plurality of compartments for storing and delivering medicinal compounds can comprise: a containing vessel made from a rigid material having an apical end and a distal end, the containing vessel defining an interior lumen, the interior lumen extending from the apical end to the distal end of the containing vessel, the interior lumen defining an opening on the distal end of the containing vessel and an opening on the apical end of the containing vessel and an inner surface within the containing vessel; a barrier connected to the apical end of the containing vessel that seals the apical end of the containing vessel; a connecting member on the apical end of the containing vessel configured to secure the barrier to the apical end of the containing vessel; a stopper assembly positioned to move from the distal end to the apical end of the containing vessel, the stopper assembly comprising: a plugging member having a distal end and a proximal end, the plugging member configured to seal the interior lumen at the distal end of the containing vessel, a flexible wall attached to the apical end of the plugging member, wherein the flexible wall and the apical end of the plugging member define an interior volume, a tab member connected to the plugging member, the tab member configured to extend away from the distal end of the plugging member and to extend into the interior volume, wherein the tab member is further configured to move when actuated to create an opening in the flexible wall; and an external volume defined by the flexible wall, the inner surface of the interior lumen, and the distal side of the barrier connected to the apical end of the containing vessel. In some embodiments, the tab member is configured to move freely in relation to the plugging member when the tab member is actuated.

A method of expunging a fluid from the external volume of some embodiments of a cartridge assembly can comprise: attaching a needle assembly to the apical end of the cartridge assembly; moving the plugging member from the distal end of the containing vessel toward the apical end of the containing vessel without actuating the tab member (e.g., while maintaining the tab member in the first position); and expunging the fluid from the external volume of the cartridge through the needle assembly. The method can further comprise inserting the cartridge assembly into a standard medicinal or dental syringe. In some embodiments, the method can comprise inserting the cartridge assembly into a syringe assembly having an opening to receive the cartridge.

A method of expunging a substance from the external volume of some embodiments of the cartridge can comprise: attaching a needle assembly to the apical end of the cartridge assembly; actuating the tab member; mixing a substance stored in the internal chamber with the substance stored in the external volume to create a mixed fluid; moving the plugging member from the distal end of the containing vessel toward the apical end of the containing vessel; and expunging the mixed substance from the external volume through the needle assembly. The method can further comprise inserting the cartridge assembly into a standard medicinal or dental syringe. In some embodiments, the method can comprise inserting the cartridge assembly into a syringe assembly having an opening to receive the cartridge.

According to some variants, a cartridge assembly with a plurality of compartments for storing and delivering medicinal compounds can comprise: a containing vessel made from a rigid material having an apical end and a distal end, the containing vessel defining an interior lumen, the interior lumen extending from the apical end to the distal end of the containing vessel, the interior lumen defining an opening on the distal end of the containing vessel and an opening on the apical end of the containing vessel and an inner surface within the containing vessel; a barrier assembly connected to the apical end of the containing vessel that seals the apical end of the containing vessel, the barrier assembly comprising: a barrier member configured to seal the apical end of the containing vessel, a flexible wall attached to the distal side of the barrier member, wherein the flexible wall and the distal side of the plugging member define an interior volume, and a tab member connected to the barrier, the tab member configured to extend from beyond the apical end of the plugging member and to extend into the interior volume, wherein the tab member is further configured to move from a first position to a second position when actuated to create an opening in the flexible wall; a connecting member on the apical end of the containing vessel configured to secure the barrier to the apical end of the containing vessel; a plugging member configured to seal the distal end of the containing vessel, the plugging member positioned to move from the distal end to the apical end of the interior lumen at the distal end of the containing vessel; and an external volume defined by the flexible wall, the inner surface of the interior lumen, and the apical end of the plugging member. In some embodiments, the tab member is configured to move freely in relation to the plugging member when the tab member is actuated.

A method of expunging a fluid from the external volume of some embodiments of a cartridge assembly can comprise: attaching a needle assembly to the apical end of the cartridge assembly; moving the plugging member from the distal end of the containing vessel toward the apical end of the containing vessel without actuating the tab member (e.g., while maintaining the tab member in the first position); and expunging the fluid from the internal volume of the cartridge through the needle assembly. The method can further comprise inserting the cartridge assembly into a standard medicinal or dental syringe. In some embodiments, the method can comprise inserting the cartridge assembly into a syringe assembly having an opening to receive the cartridge.

A method of expunging a fluid from the external volume of some embodiments of a cartridge assembly can comprise: attaching a needle assembly to the apical end of the cartridge assembly; actuating the tab member; mixing a substance stored in the internal chamber with a substance stored in the external volume to create a mixed substance; moving the plugging member from the distal end of the containing vessel toward the apical end of the containing vessel; and expunging the mixed fluid from the internal lumen through the needle assembly. The method can further comprise inserting the cartridge assembly into a standard medicinal or dental syringe. In some embodiments, the method can comprise inserting the cartridge assembly into a syringe assembly having an opening to receive the cartridge.

A method of manufacturing a cartridge assembly having two or more compartments, can comprise: selecting a hollow containing vessel made of a rigid material, wherein the containing vessel has an apical end and a distal end and an inner lumen between the apical end and the distal end of the containing vessel, the inner lumen comprising an inner wall; manufacturing a stopper assembly, wherein manufacturing the stopper assembly comprises: selecting a stopper made of a material suitable for sealing the distal end of the inner lumen, attaching a flexible wall to the apical side of said stopper, wherein said flexible wall and stopper define a sealed internal volume, filling the internal volume with a first medicinal compound, and inserting a tab into the stopper and positioning said tab to extend from the internal volume to a first position beyond the distal end of the stopper, said tab connected to the stopper to permit movement of the tab from the first position to a second position relative to the stopper; inserting the stopper assembly into the distal end of the containing vessel so that the stopper seals the distal end of the containing vessel; filling the external volume defined by the flexible wall and the inner wall of the inner lumen of the containing vessel with a second medicinal compound; applying a barrier on the apical end of the containing vessel; and using a connecting member to secure the barrier to the apical end of the containing vessel to hold the barrier in place and limit leakage of the second medicinal compound from the apical end of the containing vessel. In some embodiments, actuating the tab from the first to the second position can create fluid communication between the volume containing the first medicinal compound and the volume containing the second medicinal compound. In some embodiments, the first medicinal compound is sodium bicarbonate. In some embodiments, the second medicinal compound is Lidocaine 2% with 1:100,000 epinephrine. In some embodiments, the second medicinal compound is Kenalog/Orabase.

A method of manufacturing a cartridge assembly having two or more compartments can comprise: creating or purchasing a hollow containing vessel made of a rigid material, where in the containing vessel has an apical end and a distal end and an inner lumen extending from the apical end to the distal end, the inner lumen defining an inner surface; manufacturing a barrier assembly, wherein manufacturing the barrier assembly comprises: creating a barrier made of a material suitable for sealing the apical end of the containing vessel, attaching a flexible wall to the distal side of said barrier, wherein said flexible wall and said barrier define a sealed internal volume, filling the internal volume with a first medicinal compound, and inserting a tab into the barrier and positioning said tab to extend from the internal volume to a first position beyond the proximal end of the barrier, said tab connected to the stopper to permit movement of the tab from the first position to a second position relative to the barrier; inserting the barrier assembly into the apical end of the containing vessel so that the barrier seals the apical end of the containing vessel; using a connecting member to connect said barrier to the apical end of the containing vessel and to hold the barrier in place and to limit leakage of the first medicinal compound from the apical end of the containing vessel; filling the external volume defined by the flexible wall and the inner surface of the inner lumen of the containing vessel with a second medicinal compound; and inserting a stopper into the distal end of the containing vessel. In some embodiments, actuating the tab from the first to the second position can create fluid communication between the volume containing the first medicinal compound and the volume containing the second medicinal compound. In some embodiments, the first medicinal compound is sodium bicarbonate. In some embodiments, the second medicinal compound is Kenalog/Orabase. In some embodiments, the second medicinal compound is Lidocaine 2% with 1:100,000 epinephrine.

According to some variants, a method of using a multivolume cartridge design with at least two compartments can comprise: selecting a cartridge with two or more internal compartments, said cartridge having one or more tab assemblies configured to provide fluid communication between the two or more internal compartments when actuated, at least one of said the tab assemblies is configured to move from a first position to a second position when actuated; determining whether to actuate the one or more tab assemblies to mix the components in the two or more internal compartments; and connecting an access member to the apical end of the cartridge. In some embodiments, the method further includes actuating the one or more tab assemblies. The method can include mixing the components of two or more of the internal compartments. In some embodiments, the method further comprises attaching a plunger to the distal end of the cartridge. The method can include administering the at least a portion of the contents of the cartridge to a patient. In some embodiments, the access member comprises a needle. According to some variants, the method further comprises inserting the cartridge into a syringe.

### BRIEF DESCRIPTION OF THE DRAWINGS

The above-mentioned features and other features of the disclosure are further described below with reference to the drawings of specific embodiments. The specific embodiments shown in the drawings are intended to illustrate, but not limit the disclosure.

FIG. 1 is a perspective view of a cartridge assembly without an internal enclosure.

FIG. 2 is a perspective view of a cartridge assembly with an internal enclosure in the distal end of the cartridge assembly.

FIG. 3 is an elevated side view of one embodiment of a stopper assembly with an attached interior enclosure, showing a tab assembly configuration where the tab is a strip tab with a hole that may line up with a hole in the wall of the interior enclosure.

FIG. 3A is a cross-section schematic view of the interior enclosure wall and tab assembly through the line 3A-3A in FIG. 3, showing the tab assembly in the closed position.

FIG. 3B is a cross-section schematic view of the interior enclosure wall and tab assembly through the line 3B-3B in FIG. 3, showing the tab assembly in the open position.

FIG. 3C is an elevated side view of one embodiment of a stopper assembly with an attached interior enclosure, showing a tab assembly configuration where the tab is a strip tab with a hole that may line up with a hole in the wall of the interior enclosure.

FIG. 3D is a cross-section schematic view of the interior enclosure wall and tab assembly through the line 3D-3D in FIG. 3C.

FIG. 4 is a perspective view of one embodiment of a stopper assembly with an attached interior enclosure, showing a tab assembly configuration where the tab is a strip tab which can slide along a rail within the interior enclosure.

FIG. 5 is a perspective view of one embodiment of a stopper assembly with an attached interior enclosure, showing a tab assembly configuration where the tab is a strip tab with a plug configured to engage a hole in the wall of the interior enclosure.

FIG. 6 is a perspective view of one embodiment of a stopper assembly with an attached interior enclosure, showing a tab assembly configuration where the tab is a semi-rigid flexible tab with a sharp apical end.

FIG. 7 is a perspective view of a cartridge assembly with an interior enclosure and tab assembly in the apical end of the cartridge assembly.

FIG. 8A is an elevated view of a syringe housing.

FIG. 8B is an elevated view of a disassembled syringe assembly, with a syringe housing, an embodiment of a cartridge and an embodiment of a needle assembly.

FIG. 8C is an elevated view of the syringe assembly of FIG. 8B in an assembled configuration.

### DETAILED DESCRIPTION OF SPECIFIC EMBODIMENTS

The disclosure relates to a multivolume cartridge assembly configured to store separately two or more medicinal compounds and administer one or more of those compounds to a patient. Although the multivolume cartridge assembly will be described with reference to the above-identified figures in the context of medical and dental medical devices, the features of the multivolume cartridge assembly can provide advantages in other applications as well.

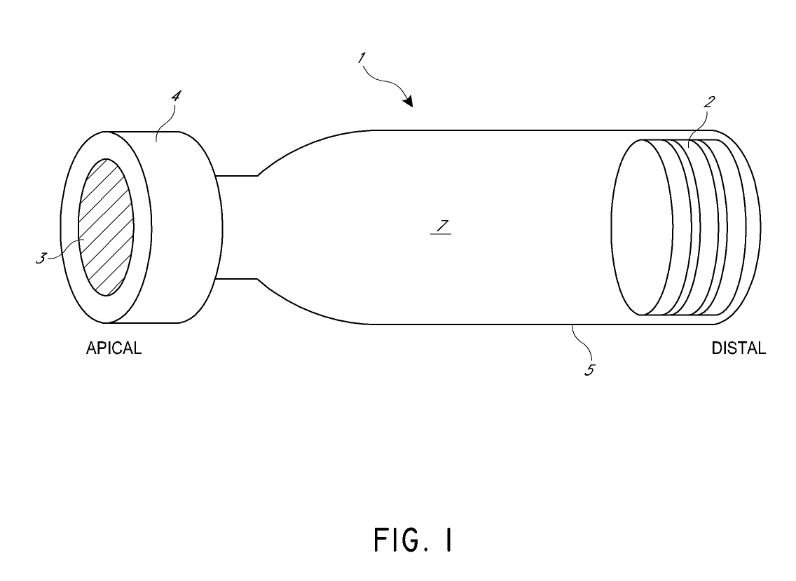

Referring to FIG. 1, a cartridge assembly 1 comprises a containing vessel 5 with an apical end (from which the contents are expunged) and a distal end. The containing vessel 5 can be sized and shaped to fit within or otherwise interface with a standard medicinal or dental syringe assembly. In some embodiments, the containing vessel 5 is approximately 0.25 inches in diameter. In some embodiments, the containing vessel 5 is approximately 2.5 inches long. In some embodiments, the length of the containing vessel 5 is less than or equal to about 4 inches and/or greater than or equal to 1 inch. In some embodiments, the diameter of the containing vessel 5 is less than or equal to about 1 inch and/or greater than or equal to about 0.2 inches. In some embodiments, the containing vessel 5 is constructed of a rigid material. In some embodiments, the rigid material is transparent. The rigid material could be glass, plastic or some other suitable material or combination of materials. The distal end of the containing vessel 5 has an opening which can be sealed with a stopper 2 constructed of rubber or some other material suitable for sealing an enclosure, such as silicone rubber or butyl rubber. The distal end of the containing vessel 5 and the stopper 2 can be constructed such that a standard medicinal or dental plunger (e.g., the plunger of a syringe assembly) can engage with the distal end of the containing vessel (e.g., a harpoon of the plunger can engage with the stopper 2). The opening on the apical end can be sealed with a barrier 3 constructed of silicone or some other material suitable for sealing an enclosure, such as latex. The barrier 3 can be held in place with a clamp 4. The apical end of the containing vessel 5 can be constructed such that a standard medicinal or dental needle assembly can be connected with or otherwise interfaced with the apical end of the containing vessel 5. The volume 7 defined by the walls of the containing vessel 5, the stopper 2 and barrier 3 can be filled with a medicinal compound. In some embodiments, the volume 7 of the containing vessel 5 can be between about 1.7-1.8 ml. In some embodiments, the volume 7 is less than or equal to about 5 ml and/or greater than or equal to about 0.75 ml.

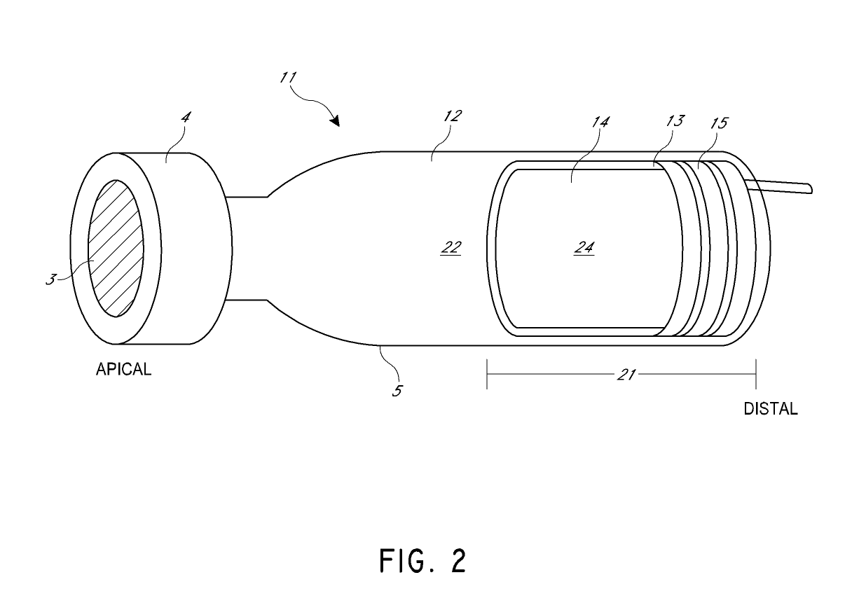

FIG. 2 illustrates a multivolume cartridge assembly 11 in which a first compound 24 and a second compound 22 can be stored. The multivolume cartridge assembly 11 comprises a containing vessel 5 with an apical end and a distal end. In an embodiment, the cartridge assembly 11 further comprises a stopper assembly 21 in the distal end of the cartridge assembly 11. The stopper assembly 21 comprises an internal enclosure 14 defined by a flexible wall 13 and a stopper 15. The stopper assembly can further comprise a tab 16. One advantage of having an enclosure internal to the containing vessel 5 is that the overall size of the container 5 can be matched to the size of standard, single-volume carpules.

An opening on apical end of the multivolume cartridge assembly 11 can be sealed with a barrier 3 constructed of silicone or some other material suitable for providing a fluid and gas seal, such as latex. The barrier 3 can be held in place by a clamp 4 of some other means suitable for retaining the barrier 3 in its sealed position. As illustrated in FIG. 2, the multivolume cartridge assembly 11 provides for two separate enclosures within the cartridge. An internal enclosure 14 can be defined by a flexible wall 13 and a stopper 15. An external enclosure 12 can be defined by the interior of the walls of the containing vessel 5, the exterior of the flexible wall 13, and the inside surface of the barrier 3. A first compound 24 can be stored in the internal enclosure 14, while a second compound 22 can be stored in the external enclosure 12.

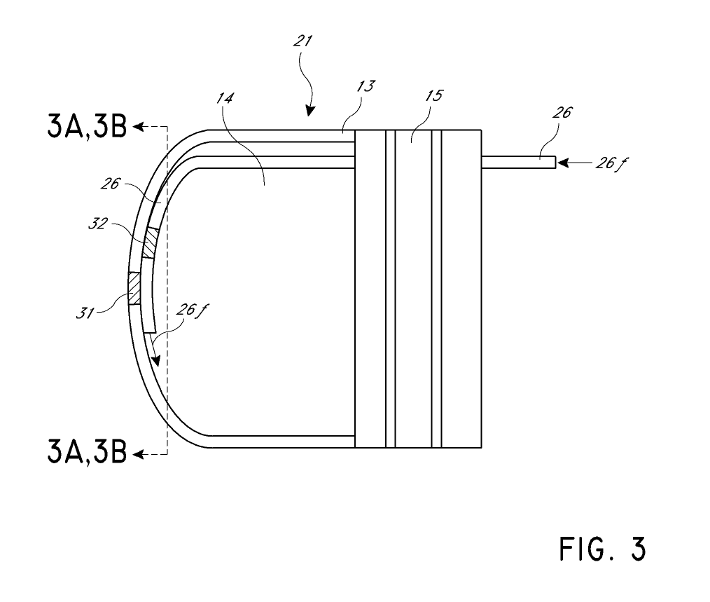

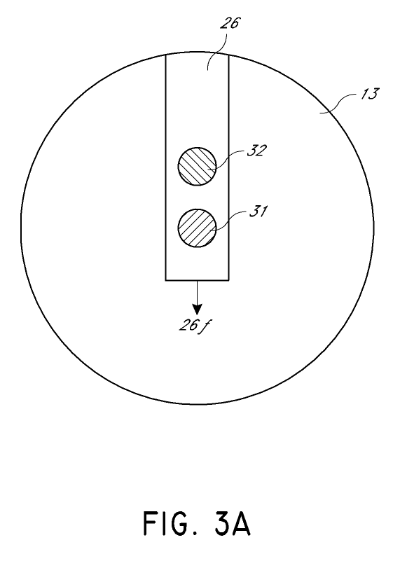

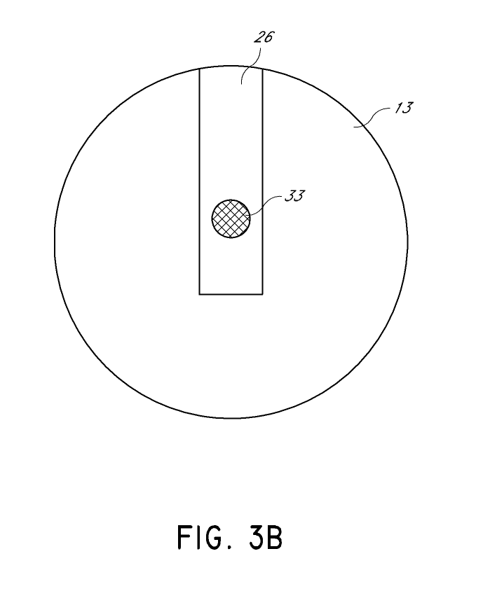

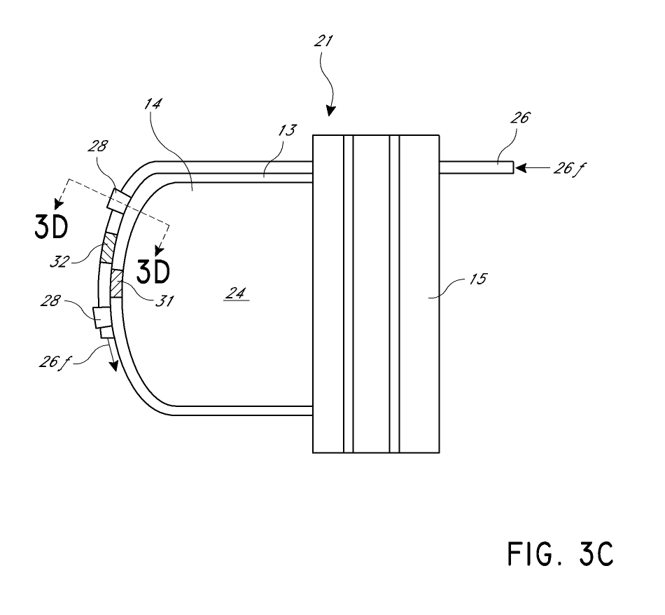

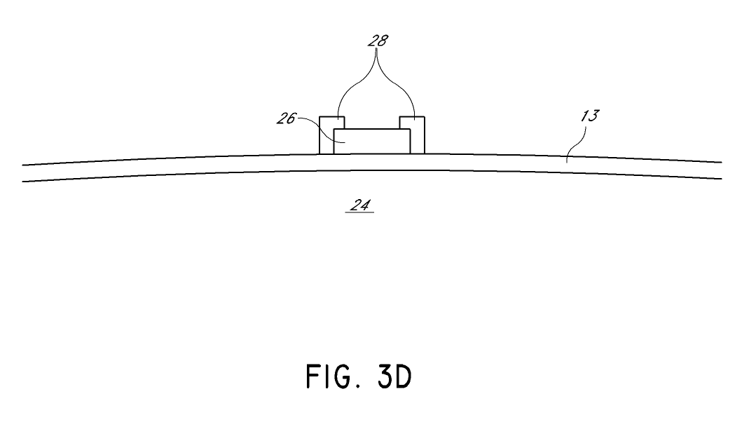

In some embodiments, a tab 16 can be coupled with the stopper 15 and/or with the containing vessel 5 to permit a first compound 24 to be mixed with a second compound 22. In an embodiment, as illustrated in FIG. 3, the stopper assembly 21 comprises a small strip tab 26 with at least one hole 32 in it. The flexible wall 13 of the internal enclosure 14 also has at least one hole 31 in it. Upon pushing the tab 26 with force 26f, the at least one hole 32 can line up with the at least one hole 31, thus creating at least one opening between the internal enclosure 14 and the external enclosure 12. FIGS. 3A and 3B illustrate from the perspective of cut-plane 3A-3A (e.g., 3B-3B) in FIG. 3 how force 26f can be used to line up the at least one hole 32 on the strip tab 26 with the at least one hole 31 in the flexible wall 13. FIG. 3B shows, once the at least one hole 32 is lined up with the at least one hole 31, at least one opening 33 is formed in the flexible wall 13. Opening 33 can thus create a fluid connection between the internal enclosure 14 and the external enclosure 12, which can allow a first compound 24 to mix with a second compound 22. It can be appreciated that a similar embodiment of the stopper assembly 21 to the one shown in FIG. 3 could be constructed so that at least one hole in the strip tab 26 could line up with the at least one hole 31 in the flexible wall 13 upon the application of a pulling force instead of a pushing force. In some embodiments, the first compound 24 is sodium bicarbonate. In some embodiments, the second compound is Lidocaine 2% with 1:100,000 epinephrine.

Although the strip tab 26 shown in FIGS. 3-5 is located on the inside of the internal enclosure 14, it could also be located on the outside of the internal enclosure 14 and operate in much the same manner. FIG. 3C illustrates an embodiment of the stopper assembly 21 wherein the strip tab 26 goes through the stopper 15 and follows along the outer surface of the flexible wall 13. FIG. 3C also shows rails 28, which can be used to guide the strip tab 26 along the surface of the flexible wall 13. An embodiment of the rails 28 is shown in FIG. 3D, which is a cross-sectional view of the tab 26, rails 28, and flexible wall 13 along cut-plane 3D-3D in FIG. 3C. The rails 28 can also be used in embodiments as shown in FIG. 3, where the strip tab 26 is located on the inside of the interior enclosure 14.

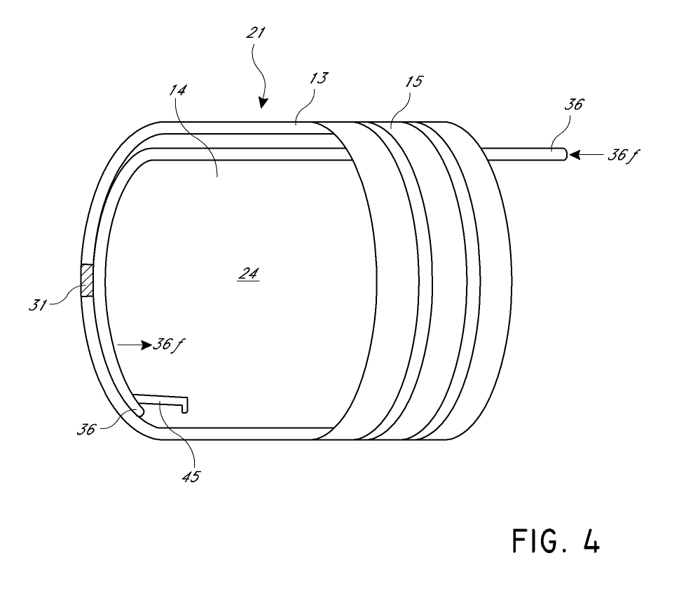

FIG. 4 illustrates another embodiment of stopper assembly 21. The stopper assembly 21 comprises a tab 36, which can be constructed to slide along a small rail 45. Thus, when a pulling force 36f is exerted on the tab 36, the tab 36 pulls away from the flexible wall 13 and exposes at least one hole 31 in the flexible wall 13. A similar embodiment of stopper assembly 21 could provide a similar function without the use of a rail 45. Upon uncovering at least one hole 31 in the flexible wall 13, the internal enclosure 14 and the external enclosure 12 can be in fluid communication, thus allowing for mixing of a first compound 24 and a second compound 22.

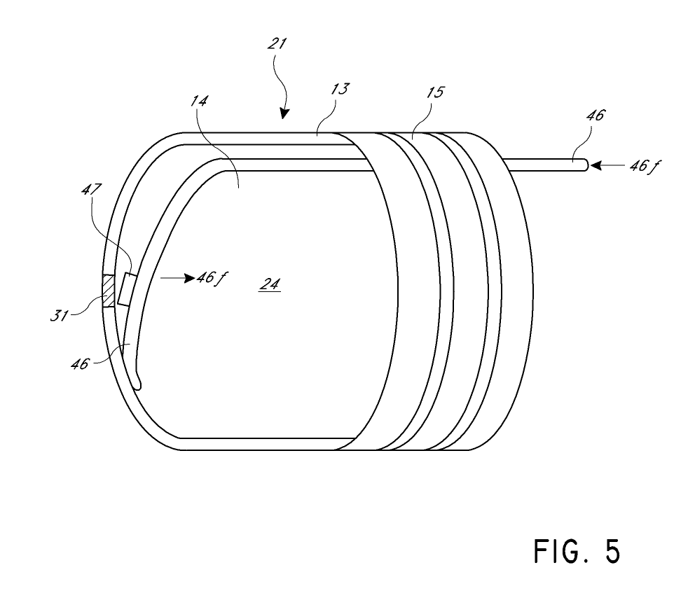

Another embodiment of stopper assembly 21 is illustrated in FIG. 5. The stopper assembly 21 comprises a tab 46 with at least one attached plug 47. The at least one plug 47 can be sized so as to engage with at least one hole 31 in the flexible wall 13. When the at least one plug 47 is engaged with at least one hole 31, a seal can be formed that can prevent the passage of liquid and gas in or out of the internal enclosure 14. Upon the application of a pulling force 46f, the plug 47 could disengage from the at least one hole 31, thus creating at least one opening in the flexible wall 13. This opening could allow for fluid communication between the internal enclosure 14 and the external enclosure 12. This opening could also allow for mixing of a first compound 24 with a second compound 22. It can also be appreciated that a similar embodiment to the one shown in FIG. 5 could be constructed so that at least one plug on the tab 46 could disengage from the at least one hole 31 in the flexible wall 13 upon a pushing force instead of a pulling force.

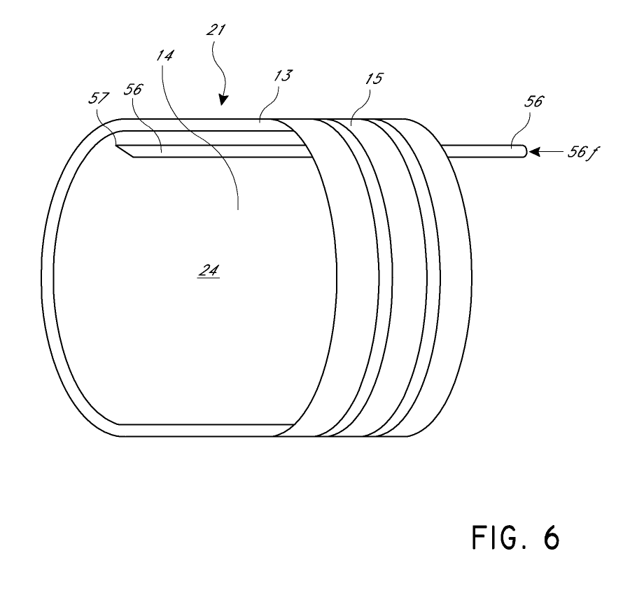

FIG. 6 illustrates another embodiment of stopper assembly 21. The stopper assembly 21 comprises a semi-rigid flexible tab 56 with a sharpened end 57. Upon application of a pushing force 56f, the sharpened end 57 of the tab 56 could puncture the flexible wall 13 of the internal enclosure 14. This puncture could create an opening in the flexible wall 13. Such an opening could allow for communication between the internal enclosure 14 and the external enclosure 12 and could allow for mixing of a first compound 24 with a second compound 22.

In some embodiments, the flexible wall 13 of the stopper assembly 21 can be configured to rupture or otherwise allow fluid communication between the internal enclosure 14 and external enclosure 12 when pressure is exerted on the distal end of the stopper 15. For example, the flexible wall 13 could be configured to rupture under increased pressure as the stopper 15 is pushed toward the apical end of the containing vessel 5. In some configurations, the pressure on the stopper 15 could be provided by a user's thumb. In some configurations, the pressure on the stopper 15 could be provided by a plunger engaged with the stopper 15.

After mixing a first compound 24 with a second compound 22, the user of the multivolume cartridge assembly 11 could place the cartridge assembly 11 into a syringe and administer the mixed solution to a patient. In some embodiments, it is not necessary to mix the contents of the interior enclosure 14 with the contents of the external enclosure 12 before, during, or after movement of the stopper 2, 15. For example, the user of the multivolume cartridge 11 can also choose to not mix a first compound 24 with a second compound 22. In some instances, the user could refrain from utilizing the tab of stopper assembly 21 and could simply insert the cartridge assembly 11 into a syringe and administer only the second compound 22 to the patient. In some instances, the first compound 24 contained in interior enclosure 14 could remain in the interior enclosure 14 and the user could decide not to expunge the first compound 24 from the multivolume cartridge assembly 11. Further, in some instances, the multivolume cartridge assembly 11 could be used with patients who do not need or should not receive (e.g., due to allergies) the first compound 24 but do need or should receive the second compound 22. In some instances, a user of the multivolume cartridge assembly 11 could first expunge a portion of the second compound 22 using the syringe, could then utilize the tab of the stopper assembly 21 to mix the remaining portion of the second compound 22 with the first compound 24, and then could expunge the remaining mixed compounds of the cartridge assembly 11. In some instances, varying dosage mixtures (e.g., ratios of the compounds 22, 24) are attainable using the same multivolume cartridge assembly 11.

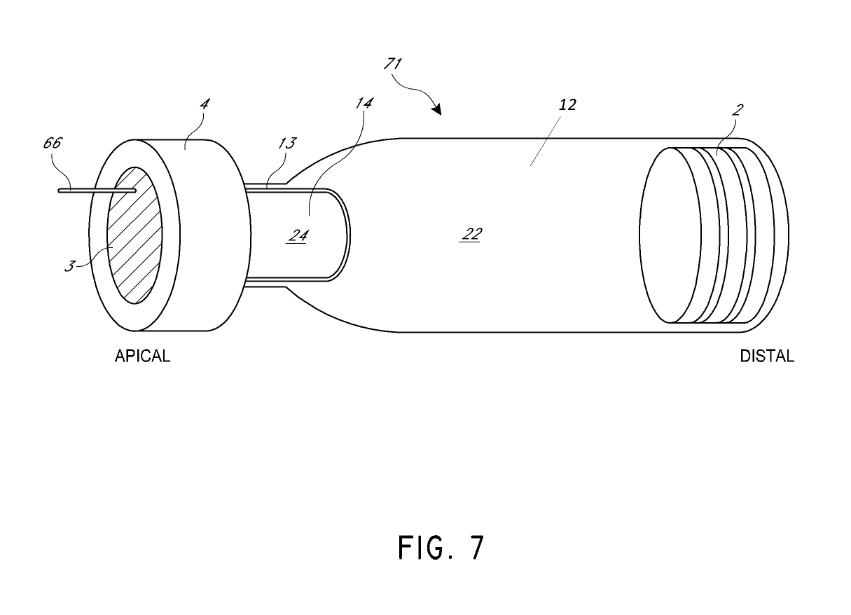

FIG. 7 illustrates a multivolume cartridge assembly 71 wherein the internal enclosure 14 is formed in the apical end of the cartridge assembly 71. In some an embodiment, a tab 66 could be positioned in the apical end of cartridge assembly 71. Each of the tab configurations illustrated in FIGS. 3-6 could be utilized in an apical enclosure embodiment as shown in FIG. 7 to allow for fluid communication between the internal enclosure 14 and the external enclosure 12.

The tabs 16, 26, 36, 46, 56, 66 can be configured to move and/or actuate independently from (e.g., without moving) the stopper 15, barrier 3, plunger, and/or containing vessel 5 to which they are coupled. For example, the tabs 16, 26, 36, 46, 56, 66 can be pushed and/or pulled in the apical and/or distal direction without moving the stopper 2, 15, barrier 3, or containing vessel 5. In some embodiments, mixing the first compound 24 with the second compound 22 without moving the stopper 2, 15 or barrier 3 can reduce or eliminate an increase in pressure within the containing vessel 2 as the first and second compounds 24, 22 are mixed due to movement of the stopper 2, 15 and/or barrier 3. In some embodiments, minimizing any increase in pressure within the containing vessel 5 can reduce stresses on the barrier 3 and/or stopper 2 and can reduce the likelihood that the contents of the containing vessel 5 accidentally leak or otherwise escape the vessel 5. In some embodiments, mixing the first compound 24 with the second compound 22 without moving the stopper 2, 15 or barrier 3 can increase the likelihood that the compounds 24, 22 are fully mixed before administering the mixture. For example, the compounds 24, 22 can be mixed prior to coupling a needle to the apical end of the containing vessel 2 and/or prior to coupling a plunger to the stopper 2.

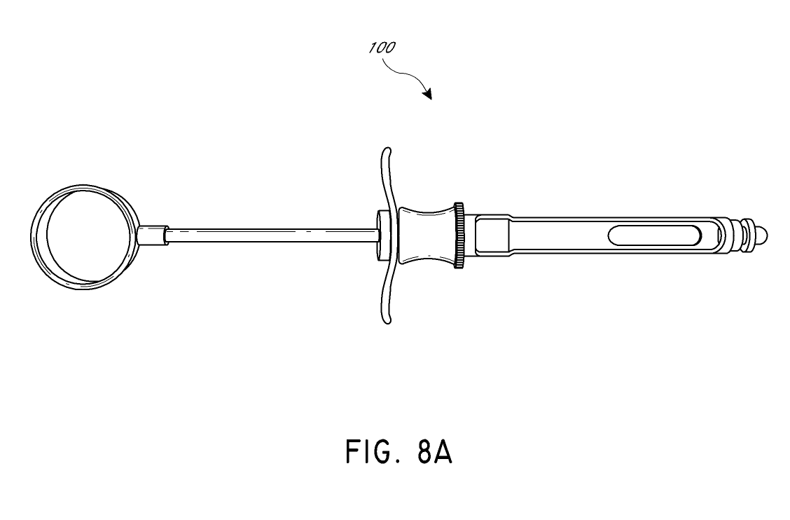

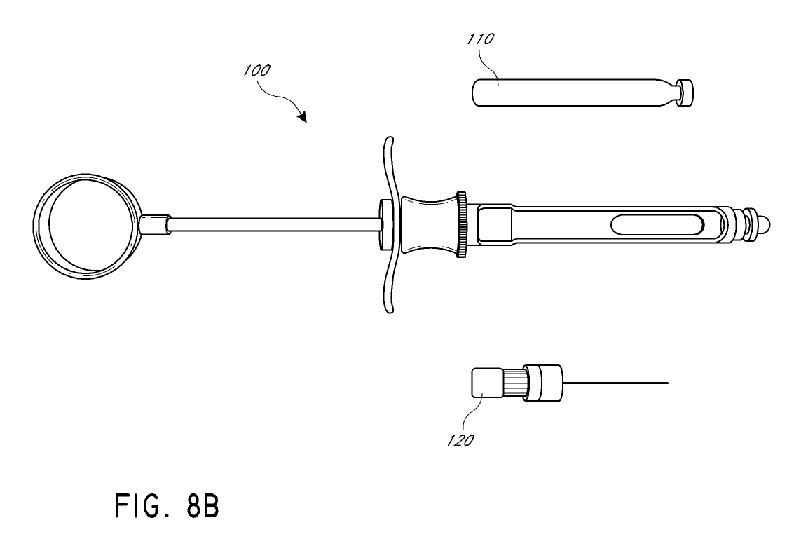

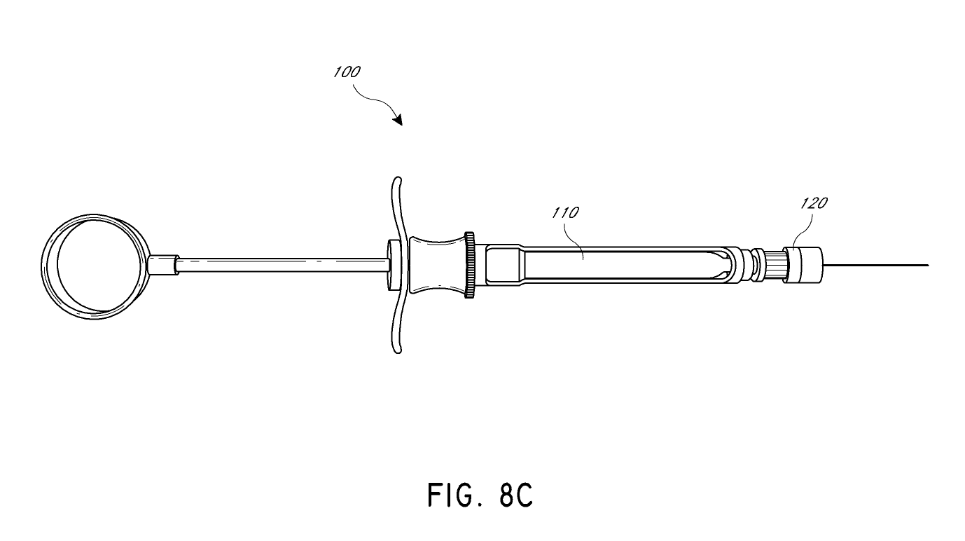

FIGS. 8A-8C illustrate an embodiment of a syringe assembly. A syringe housing 100 is shown in FIG. 8A. The syringe housing 100 can include an opening configured to receive a cartridge 110. FIG. 8B shows the syringe housing 100, and embodiments of a cartridge 110 and a needle assembly 120. In some configurations, the cartridge 110 is configured to be inserted into the syringe housing 100. The needle assembly 120 can be configured to engage with the cartridge 110 and syringe housing 100, as shown in FIG. 8C.

Although certain embodiments, features, and examples have been described herein, it will be understood by those skilled in the art that many aspects of the methods and devices illustrated and described in the present disclosure may be differently combined and/or modified to form still further embodiments. For example, other means similar to those described in the above embodiments could be used for creating an opening in the flexible wall 13. Additionally, it will be recognized that the methods described herein may be practiced in different sequences, and/or with additional devices as desired. Such alternative embodiments and/or uses of the methods and devices described above and obvious modifications and equivalents thereof are intended to be included within the scope of the present invention. Furthermore, the term “carpule” as used above could refer to medicinal and/or dental containers in many medicinal or dental applications and should not be read to limit the application of the above-described features to any single use. For example, the features described above need not be limited to containers that fit any specific syringe housing, nor should the features be limited to containers for use in syringes. Thus, it is intended that the scope of the present invention should not be limited by the particular embodiments described above, but should be determined only by the claims that follow.

What is claimed is:

1. A cartridge assembly with a plurality of compartments for storing and delivering medicinal compounds comprising:
a containing vessel made from a rigid material having an apical end and a distal end, the containing vessel defining an interior lumen, the interior lumen extending from the apical end to the distal end of the containing vessel, the interior lumen defining an opening on the distal end of the containing vessel and an opening on the apical end of the containing vessel and an inner surface within the containing vessel;
a barrier connected to the apical end of the containing vessel that seals the apical end of the containing vessel;
a connecting member on the apical end of the containing vessel configured to secure the barrier to the apical end of the containing vessel;
a stopper assembly positioned to move between the distal end and the apical end of the containing vessel, the stopper assembly comprising:
a plugging member having a distal end and a proximal end, the plugging member configured to seal the interior lumen at the distal end of the containing vessel, a flexible wall attached to the apical end of the plugging member, wherein the flexible wall and the apical end of the plugging member define an interior volume, and
a tab member connected to the plugging member, the tab member configured to extend away from the distal end of the plugging member and to extend into the interior volume, wherein the tab member is further configured to move from a first position to a second position when actuated to create an opening in the flexible wall; and
an external volume defined by the flexible wall, the inner surface of the interior lumen, and the distal side of the barrier connected to the apical end of the containing vessel.

2. The cartridge assembly of claim 1, wherein the tab member is configured to move freely in relation to the plugging member when the tab member is actuated.

3. A method of expunging a fluid from the external volume of the cartridge assembly of claim 1, the method comprising:
attaching a needle assembly to the apical end of the cartridge assembly;
moving the plugging member from the distal end of the containing vessel toward the apical end of the containing vessel while maintaining the tab member in the first position; and
expunging the fluid from the external volume of the cartridge through the needle assembly.

4. The method of claim 3 further comprising inserting the cartridge assembly into a syringe assembly having an opening to receive the cartridge.

5. A method of expunging a substance from the external volume of the cartridge assembly of claim 1, the method comprising:
attaching a needle assembly to the apical end of the cartridge assembly;
actuating the tab member to move from the first position to the second position;
mixing a substance stored in the internal chamber with the substance stored in the external volume to create a mixed fluid;
moving the plugging member from the distal end of the containing vessel toward the apical end of the containing vessel; and
expunging the mixed substance from the external volume through the needle assembly.

6. The method of claim 5 further comprising inserting the cartridge assembly into a syringe assembly having an opening to receive the cartridge.

7. A cartridge assembly with a plurality of compartments for storing and delivering medicinal compounds comprising:
a containing vessel made from a rigid material having an apical end and a distal end, the containing vessel defining an interior lumen, the interior lumen extending from the apical end to the distal end of the containing vessel, the interior lumen defining an opening on the distal end of the containing vessel and an opening on the apical end of the containing vessel and an inner surface within the containing vessel;
a barrier assembly connected to the apical end of the containing vessel that seals the apical end of the containing vessel, the barrier assembly comprising:
a barrier member configured to seal the apical end of the containing vessel, a flexible wall attached to the distal side of the barrier member, wherein the flexible wall and the distal side of the plugging member define an interior volume, and
a tab member connected to the barrier, the tab member configured to extend from beyond the apical end of the plugging member and to extend into the interior volume, wherein the tab member is further configured to move from a first position to a second position when actuated to create an opening in the flexible wall;
a connecting member on the apical end of the containing vessel configured to secure the barrier to the apical end of the containing vessel;
a plugging member configured to seal the distal end of the containing vessel, the plugging member positioned to move from the distal end to the apical end of the interior lumen at the distal end of the containing vessel; and
an external volume defined by the flexible wall, the inner surface of the interior lumen, and the apical end of the plugging member.

8. The cartridge assembly of claim 7, wherein the tab member is configured to move freely in relation to the barrier member when the tab member is actuated.

9. A method of expunging a fluid from the internal volume of the cartridge assembly of claim 7, the method comprising:
attaching a needle assembly to the apical end of the cartridge assembly;
moving the plugging member from the distal end of the containing vessel toward the apical end of the containing vessel while maintaining the tab member in the first position; and
expunging the fluid from the internal volume of the cartridge through the needle assembly.

10. The method of claim 9 further comprising inserting the cartridge assembly into a syringe assembly having an opening to receive the cartridge.

11. A method of expunging a fluid from the internal lumen of the cartridge assembly of claim 7, the method comprising:
attaching a needle assembly to the apical end of the cartridge assembly;
actuating the tab member to move from the first position to the second position;
mixing a substance stored in the internal chamber with a substance stored in the external volume to create a mixed substance;
moving the plugging member from the distal end of the containing vessel toward the apical end of the containing vessel; and
expunging the mixed fluid from the internal lumen through the needle assembly.

12. The method of claim 11 further comprising inserting the cartridge assembly into a syringe assembly having an opening to receive the cartridge.

13. A method of using a multivolume cartridge design with at least two compartments, comprising:
selecting a cartridge with two or more internal compartments, said cartridge having one or more tab assemblies configured to provide fluid communication between the two or more internal compartments when actuated, at least one of said the tab assemblies is configured to move from a first position to a second position when actuated;
determining whether to actuate the one or more tab assemblies to mix the components in the two or more internal compartments; and
connecting an access member to the apical end of the cartridge.

14. The method of claim 13, wherein the method further comprises actuating the one or more tab assemblies.

15. The method of claim 14, wherein the method further comprises mixing the components of two or more of the internal compartments.

16. The method of claim 13, wherein the method further comprises attaching a plunger to the distal end of the cartridge;

17. The method of claim 13, wherein the method further comprises administering the at least a portion of the contents of the cartridge to a patient.

18. The method of claim 13, wherein the access member comprises a needle.

19. The method of claim 13, wherein the method further comprises inserting the cartridge assembly into a syringe assembly having an opening to receive the cartridge.
